(19) United States
(12) Patent Application Publication
(10) Pub. No.: US 2005/0010451 A1
(43) Pub. Date: Jan. 13, 2005

(54) Method, system, and apparatus for clinical trial management over a communications network

(75) Inventors: Ronald G. Marks, Gainesville, FL (US); Michael Conlon, Gainesville, FL (US); Phillip Padgett, Gainesville, FL (US)

Correspondence Address: AKERMAN SENTERFITT, P.O. BOX 3188, WEST PALM BEACH, FL, 33402-3188, US

(73) Assignee: University of Florida, Gainesville, FL (US)

(21) Appl. No.: 10/840,870

(22) Filed: May 7, 2004

Related U.S. Application Data
(60) Provisional application No. 60/468,912, filed on May 8, 2003.

(51) Int. Cl. G06F 17/60; G09B 19/18; G09B 19/00; A61B 10/00

(52) U.S. Cl. 705/3; 434/118

(57) ABSTRACT

A centralized, online clinical study system configured to coordinate aspects of a clinical study can include a training module configured to evaluate whether users have completed training for selected workflows for the clinical study that are available from the clinical study system, and to provide training to registered users for the selected workflows. The system also can include a financial engine configured to determine when a participating site meets or exceeds a milestone of the clinical study and to initiate a payment to the participating site. Further, the system can include a module configured to receive and verify clinical research data and to designate the verified clinical research data as official source data for the clinical study.

### CROSS-REFERENCE TO RELATED APPLICATIONS

This application claims the benefit of U.S. Provisional Application No. 60/468,912, filed in the United States Patent and Trademark Office on May 8, 2003, the entirety of which is incorporated herein by reference.

### BACKGROUND OF THE INVENTION

1. Technical Field

This invention relates to the coordination of clinical studies over a communications network.

2. Description of the Related Art

The pharmaceutical, biotechnology, and medical device industries conduct extensive clinical trials to evaluate newly developed products. Although each clinical trial can be somewhat unique, each clinical trial also must follow a standard protocol which provides a standard method of conduct to evaluate the efficacy and safety of the new products. Clinical trial conduct is critical for the Federal Drug Administration (FDA) to decide whether to approve new products.

Thousands of clinical trials are performed each year using manual, paper-driven processes which have been used for many years. Although a number of vendors offer electronic data capture (EDC) products, these products typically are limited to capturing data from study subjects and are used purely within the domain of each individual investigator or participating office. That is, data captured using these products is not shared from office to office. This is due, in large part, to the reality that the majority of clinical trial procedures remain manual and paper driven in nature.

Presently, multiple vendors are forming partnerships in an effort to bring each vendor's technology solution together to produce a more complete and integrated electronic clinical trial. This strategy, however, has disadvantages. One such disadvantage is that typically vendors are reluctant to share information with one another and to coordinate the solution in a central location that allows access by all parties.

Another disadvantage is that in order to provide an adequate solution that is not piecemeal in nature, only a small portion of each vendor's technology may be needed. Thus, a significant amount of time and developmental effort can be required to link the disparate components of each vendor's technology to function as a cohesive and operational system. Further, the management of a clinical trial is spread among a plurality of disparate systems which are neither managed nor maintained in a centralized fashion.

For example, if one company provides complete fiscal management of a clinical trial, that component can be integrated into a larger system to provide for investigator compensation. As this individual component was not designed to work within a comprehensive system, the fiscal management component would not be configured to interact with other components. For instance, the fiscal management component would be unable to provide a separate verification back to an EDC vendor indicating that payment has been made—an extra, but nonetheless necessary step that would otherwise be performed manually.

Accordingly, the strategy of integrating technology components from various vendors of a partnership likely will suffer from the same inefficiencies identified in the current EDC market. While some vendors may communicate and share information to provide a minimum level of enhanced services, what is needed is central management of the clinical trial process.

### SUMMARY OF THE INVENTION

The inventive arrangements disclosed herein provide an architecture through which clinical trials can be managed, administered, and performed. The inventive arrangements disclosed herein utilize computer communication networks, such as the Internet and/or World Wide Web, to provide a system in a central, networked location for managing documentation, prescriptions, financial matters, and other tasks, to be described herein, which are necessary when conducting clinical trials or studies. By using a system which provides for centralized management of most, if not all, aspects of the clinical trial process, benefits accrue such as improved security, the ability to monitor clinical trial procedures in real time, improved accuracy, improved timeliness of study, reduced time required for conducting clinical trials, reduced cost, as well as improved patient safety.

The present invention can provide for randomization, protocol implementation and amending, document tracking, adverse event reporting, site monitoring, report generation, and data analysis. Other activities also can be conducted or coordinated electronically over the computer communications network such as controlling investigator participation, providing a Web-based research pharmacy for managing and distributing medications, and making and recording electronic financial payments to study sites. Notably, the present invention allows electronic payments to be made based upon the timeliness and the quality of collected data. The electronic payment functionality further integrates with electronic data capture (EDC) systems and/or provide payment notification to such systems.

Another aspect of the present invention is that the various forms, reports, and functions disclosed herein can be provided as templates. Accordingly, a new study to be managed online can be configured quickly using these templates. The various templates provide reusable and customizable modules which can be used from one study to the next.

One embodiment of the present invention can include a method of coordinating a clinical study at a plurality of participating sites within a centralized, online clinical study system. The method can include logging a user onto the clinical study system, comparing workflows of the clinical study that are available through the clinical study system with workflows for which the user has been authorized, and restricting access to workflows of the clinical study for which the user has not been trained. Online training for a workflow or clinical trial activity for which the user has not been trained can be provided to the user.

The method also can include providing online training materials to the user, wherein the training materials are for a workflow for which the user has not been trained, testing the user regarding the content of the online training materials, and determining whether the user passed the test. If the user passed the test, the user can be authorized to access the workflow for which the user was trained. If the user does not pass the test, the method can include presenting the online training material to the user, wherein selections of the training material that relate to at least one question the user answered incorrectly are visually indicated, and retesting the user regarding the content of the online training materials. The user can be retested until a sponsor designated passing score is achieved.

Another embodiment of the present invention can include a method of coordinating a clinical study at a plurality of participating sites within a centralized, online clinical study system. The method can include configuring the clinical study system with a payment schedule of the clinical study, receiving clinical research data from participating sites via a communications network, and comparing the clinical research data with the payment schedule. If one or more criteria of the payment schedule have been met or exceeded, an electronic payment to an account designated by the participating site from which the clinical research data was received can be initiated. The amount of the payment can be established for reaching the milestone during the configuration of the online clinical study system. Further, the milestone can specify a time frame in which particular portions of clinical research data are to be received.

Another embodiment of the present invention can include a method of coordinating a clinical study at a plurality of participating sites within a centralized, online clinical study system. The method can include receiving clinical research data pertaining to the clinical study, receiving a user input indicating that the information is complete, and causing a summary of the clinical research data to be presented, whereby the user having provided the clinical research data can verify the accuracy of the clinical research data. The method also can include receiving an additional user input indicating that the clinical research data is accurate, storing the clinical research data within a data store of the clinical study system, and designating the stored clinical research data as official electronic source data for the clinical study. Notably, prior to the step of receiving an additional user input, the method also can include receiving a user input indicating that the clinical research data is inaccurate and receiving user corrections to the clinical research data.

Yet another embodiment of the present invention can include a centralized, online clinical study system configured to coordinate aspects of a clinical study. The clinical study system can include a training module configured to evaluate whether users have completed training available from the clinical study system that pertains to selected workflows for the clinical study, and to provide training to registered users for the selected workflows. The system also can include a financial engine configured to determine when a participating site meets or exceeds a milestone of the clinical study based upon clinical research data received via a communications network from the participating site. The financial engine can initiate a payment to an account designated by the participating site when the participating site meets or exceeds the milestone of the clinical study.

The system further can include an audit engine configured to receive clinical research data and present a summary of the clinical research data to the user having provided the clinical research data for verification. The audit engine can store the clinical research data and designate the stored clinical research data as official source data for the clinical study. An institutional review board engine also can be provided as part of the system. The institutional review board engine can be configured to determine whether a potential test site has met requirements for participating within the clinical study.

Also included in the system can be a query management engine configured to open, close, and answer queries relating to items of clinical research data. The query management engine routes queries to investigators that originally entered the data item that is the subject of the query and further prevents a user from answering a query without first correcting the subject data item.

In another embodiment, the online clinical study system can include at least one case report form that can be transmitted over a communications link to a participating site computer system. One or more data fields of the case report form can be linked with a portion of a protocol of the clinical study explaining those data entry fields. The system also can include an adverse event management engine. The adverse event management engine can electronically notify a third party of selected adverse events. For example, such a notification or notifications can be electronically sent to the Food and Drug Administration.

Still another embodiment of the present invention can include a machine readable storage programmed for causing a machine to perform the various steps described herein.

### BRIEF DESCRIPTION OF THE DRAWINGS

There are shown in the drawings embodiments which are presently preferred, it being understood, however, that the invention is not limited to the precise arrangements and instrumentalities shown.

FIG. 1 is a schematic diagram illustrating one embodiment of a system for managing aspects of a clinical trial over a communications network in accordance with the inventive arrangements disclosed herein.

FIG. 2 is a schematic diagram illustrating another embodiment of a system for managing aspects of a clinical trial over a communications network in accordance with the inventive arrangements disclosed herein.

FIG. 3 is a flow chart illustrating a method of training users for workflows for a clinical study in accordance with another embodiment of the present invention.

FIG. 4 is a flow chart illustrating a method of electronic source document verification for use within a clinical study in accordance with another embodiment of the present invention.

FIG. 5 is a flow chart illustrating a method of processing financial transactions within a clinical study in accordance with another embodiment of the present invention.

FIG. 6 is a flow chart illustrating an example of an investigator and/or sponsor selecting a particular workflow in accordance with one embodiment of the present invention.

FIGS. 7A and 7B, taken together, are a flow chart illustrating an overview of the query functionality provided in accordance with another embodiment of the present invention.

### DETAILED DESCRIPTION OF THE INVENTION

The present invention provides a method, system, and apparatus for managing clinical trials in a networked computing environment. As such, the present invention can provide centralized management of site equipment and training, site study administration, Institutional Review Board (IRB) operation, patient eligibility, randomization, site monitoring, management and monitoring of study progress, medication dispensing, adverse event (AE) reporting, documentation of the medical summary, study security, Federal Drug Administration (FDA) required data validation, data management, and study reporting.

According to one embodiment of the present invention, a Web-based approach for managing aspects of a clinical trial is provided that allows sponsors to communicate with a centralized system using a computer system and appropriate visual and/or voice browser. Interactions with the clinical trial can be performed over a computer communications network, such as the Web, and recorded in a central data store and/or database.

As used herein, the phrase “clinical trial” or “trial” can refer to any of a variety of different testing procedures, phases, or studies relating to therapeutic and/or prophylactic drug therapies, treatments, and/or medical devices, which are suited for use with human beings, animals, microorganisms, and the like. The present invention also can be used in the context of observational studies, registry studies, outcome studies, consumer evaluations of products, and/or pre-clinical lab and/or animal research.

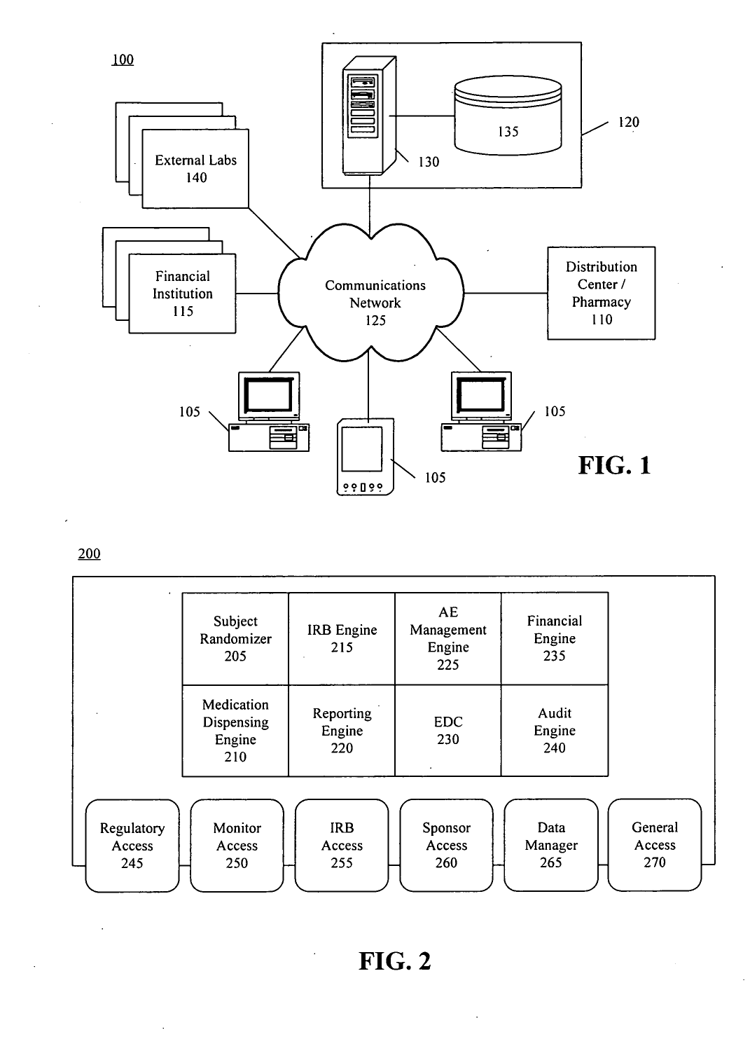

FIG. 1 is a schematic diagram illustrating one embodiment of a system for managing aspects of a clinical trial over a communications network in accordance with the inventive arrangements disclosed herein. As shown, the system 100 can include one or more test site information processing systems 105, one or more financial institutions 115, a distribution center/pharmacy (pharmacy) 110, an online clinical study system 120, and one or more external labs 140. The online clinical study system 120 can be remotely located from the financial institutions 115, the computer systems 105 at the participating or test sites, and the pharmacy 110.

The aforementioned systems or nodes can be communicatively linked via a communications network 125. That is, each node can include a computer system and/or other information processing system having suitable networking hardware, whether wired or wireless, for communicating over the communications network 125. The communications network 125 can be implemented and/or include a local area network, a wide area network, the Internet, the Public Switched Telephone Network, mobile communication and/or wireless networks, and the like.

The online clinical study system 120 can be a centralized system that includes a server 130 and a data store 135, whether a database or some other structure for storing data. While shown as single objects, it should be appreciated that the server 130 and the data store 135 can be implemented as one or more distributed storage devices and/or computer systems, each being communicatively linked with one another as well as the communications network 125, and each being part of the online clinical study system 120.

In any case, the server 130 can execute one or more applications, programs, and/or scripts for coordinating aspects of a clinical study in an online fashion. Data required by the server 130 can be stored within the data store 135. The server 130 can control aspects of a clinical study including, but not limited to, subject randomization, performing institutional review board certifications, performing adverse event monitoring, dispensing medications, reporting results, and validating received data. The server 130 further can initiate financial payments for participating sites once one or more criteria of a payment schedule has been met or exceeded and provide online training.

The data store 135 can include data for subjects participating in the clinical study, clinical research data for each subject collected during the clinical study, information pertaining to the participating sites, and the like. The data store 135 also can include documentation and procedural information pertaining to the clinical study. For example, case report forms, informed consent forms, and the study protocol can be stored online and, thus, be available for printing and/or viewing. With respect to the fields of the case report form, as it is in electronic format such as a Web page, each field on each case report form can be linked to the corresponding explanation in the protocol for that field. The linking of fields with protocol explanations can provide instant access to protocol information as the data is being entered. This feature provides complete protocol access to study constituents as they do their clinical trial work.

The data store 135 further can include documentation paperwork for study medications shipped to the participating site including how those medications are to be distributed to subjects. Training materials also can be provided within the data store 135. The training materials, or content, can include audio, text, graphics, and/or video which can be accessed by clinical trial participants for training at that participant's time of choosing. Results of the training can be saved and documented in the data store 135 in accordance with FDA 21 C.F.R. Part 11 guidelines.

The various documents stored within data store 135 can be updated from time to time as required such that the most current version of each document is available. Amendments to each respective electronic document can be indicated if desired.

In operation, the online clinical study system 120 can receive clinical research data from one or more information processing systems 105 located at one or more sites having been approved for participating in a clinical study. Additionally, the online clinical study system 120 can receive clinical research data, for example test results, electronically from external labs or testing facilities 140. The clinical research data can be stored in the data store 130 and processed as described herein. The online clinical study system 120 further can be programmed with a payment schedule. The payment schedule can specify the criteria that is to be met by a participating site before a payment is made to that test site. Notably, while a single general payment schedule can be provided, it should be appreciated that each individual participating site can have its own payment schedule thereby allowing the online clinical study system 120 to initiate payments according to individualized criteria corresponding to each participating site, rather than according to a single generalized schedule.

In one embodiment, the payment schedule can specify incentives and/or penalties for meeting or failing to meet one or more criteria respectively. For example, the criteria may specify that a participating site is to receive a payment of $500.00 per study subject if a target number of study subjects is registered by a particular deadline. The criteria can specify an incentive that would lead to a payment of $700.00 per study subject, for example, if the target number of study subjects is registered well before the deadline. The criteria further can specify a penalty or disincentive that would lead to a payment of only $300.00 per study subject if the participating site registered the target number of study subjects, but missed the stated deadline. It should be appreciated that the examples described herein are provided for purposes of illustration and are not to be construed as limiting the scope of the present invention.

Accordingly, the online clinical study system 120 can monitor the received data and compare the received clinical research data with the criteria specified in the payment schedule(s). If one or more of the participating sites meets or exceeds the criteria, the online clinical study system 120 can initiate a payment to the participant site. More particularly, the online clinical study system 120 can contact its financial institution 115 via the communications network 125. The online clinical study system 120 can request that money be electronically transferred from an account affiliated with the online clinical study system 120 to an account designated by the participating site that met or exceeded the criteria of the payment schedule. The designated account can be at another financial institution 115.

As patients are enrolled and as the study progresses, the clinical study system 120 further can contact the pharmacy 110, via communications network 125, to request the shipment of medication. Medication then can be shipped to clinical study patients or to the participating sites.

FIG. 2 is a schematic diagram illustrating another embodiment of a system 200 for managing aspects of a clinical trial over a communications network in accordance with the inventive arrangements disclosed herein. FIG. 2 provides an illustration of the software architecture which can execute with the server 130 of FIG. 1. As shown, the system 200 can include a subject randomizer 205, a medication dispensing engine 210, an IRB engine 215, a reporting engine 220, and AE management engine 225, an electronic data capture (EDC) engine 230, a financial engine 235, and an audit engine 240. The aforementioned components can be implemented as a plurality of application programs or as a single, more complex application program, for example within a Web site.

The aforementioned components, i.e. the processing engines and subject randomizer 205, can access, that is store and retrieve data, from a centralized data store and/or database. Alternatively, each of the respective components can include one or more individual data stores and/or databases (not shown).

As noted, the system 200 can be implemented as a Web site, for example, wherein various participating entities of a clinical trial can access the centralized Web site via appropriate interfaces, or Web pages. For instance, the system 200 can include various access pages such as a regulatory access interface 245 for regulatory personnel, a monitor access interface 250 for use by monitoring personnel, an IRB access interface 255, as well as a sponsor access interface 260 for clinical trial sponsors. The system 200 also can include a data manager interface 265 and a generalized site access interface 270.

The various interfaces or Web pages provide access to the data processing engines and functions disclosed herein. Notably, each interface can provide selective access to these functions. More particularly, limitations can be imposed such that investigator sites, that is participating medical offices, treatment centers, and the like, only have access to particular features or to a particular portion of the data. In this manner, an investigator site can access data generated by that site, while being precluded from accessing data from other investigator sites. Trial sponsors may be given broader access to an expanded feature set. In any case, each of the particular interfaces can be associated with a particular set of access rights and, therefore, provide access to selected functions and/or data sets.

It should be appreciated that the various interfaces and data processing engines and/or functions disclosed herein are not intended to limit the scope of the present invention. Rather, the inventive arrangements described herein are provided for purposes of illustration. Other embodiments are contemplated as well. For example, a unified interface can be provided wherein an accessing trial participant, upon logon, provides an identifier which associates the participant with a particular class of user having a set of access rights governing that participant's interaction with the system 200. After login, the participant can be shown one or more other pages and/or interfaces to the functions which that participant may access.

The system 200 can include one or more electronic forms which can be accessed by participating sites. Once the electronic forms are filled in, the system 200 can determine whether subjects or patients are eligible for the clinical trial. Accordingly, the investigator site can be so notified. If the subject is eligible, the subject can be automatically enrolled in the clinical trial. Accordingly, the system 200 can include subject identifying information and other medical and/or product usage data.

The subject randomizer 205 can assign a study treatment automatically once a subject is determined to be eligible for the study. The result can be automatically stored in a data store. The subject randomizer 205 can be implemented, for example, using a random number generator function. When used in conjunction with online subject eligibility checking, the subject randomizer 205 further eliminates assignment errors. In any case, randomization can be performed online in the context of a Web-based clinical trial.

The medication dispensing engine 210 provides an interface between one or more research pharmacies and the investigator or participating sites. For example, in studies where medication distribution can be delayed from assignment, the research pharmacy can be employed to provide all medication management and distribution. In this case, individual investigator sites have no responsibility for study medications. After the subject is randomized through the system 200 using the subject randomizer 205, the medication dispensing engine 210 can electronically transmit the assigned prescription to the research pharmacy for filling and delivery to the subject and/or investigator site.

If the study allows medication choices or the physician is permitted to dispense extra medications under the study protocol, the medication dispensing engine 210 can provide or render additional screens or graphical user interfaces which allow the physician to make such medication choices before the prescription is electronically forwarded to the research pharmacy.

Use of the medication dispensing engine 210 allows for all medication management at a single, well-controlled mail-order research pharmacy. The cost of mailing medications can be offset by increased efficiency of this medication distribution system. Dispensing errors can be minimized or eliminated and subjects can receive only the appropriate medications for their assigned treatment.

Alternatively, medications can be supplied to investigative sites in smaller lots so that study medication can be dispensed immediately upon subject randomization. By using smaller distributions to investigator sites and more frequent replenishments to the site, wastage in clinical supplies can be minimized.

The IRB engine 215 provides electronic approval of an investigator site before that site can begin enrolling patients into the study. The IRB engine 215 maintains the approved Informed Consent documents electronically and updates the documents as changes are made throughout the study. The IRB engine 215 ensures that the most up-to-date and correct Informed Consent document is always in use by an investigator site. The IRB engine 215 can monitor study conduct of all investigator sites as appropriate, by receiving online reports in real time of individual and cumulative AE's. Notably, the IRB engine 215 also can process annual or periodic renewals of investigator sites.

The IRB engine 215 can electronically remove an investigator site from participation in a study when appropriate, such as when a medical license for a site investigator has expired or when subject safety may be in question. When the IRB engine 215 has electronically withdrawn an investigator site, the site can no longer enroll new subjects, enter follow-up data, or access existing subject data. One exception, however, would be for sites to continue to enter follow-up data if continued medication dispensing is necessary for subject safety. Accordingly, the IRB engine 215 can access the study enrollment data within the data store and alter the data as necessary.

In another embodiment, the IRB engine 215 can process annual certification renewals for participating sites. That is, the IRB engine 215 can be provided with updated information for each participating site each year. Each site can be provided with an electronic notification to complete or update the participating sites information and provide that information back to the system 200, for example via a Web page or other electronic communication such as electronic mail. The IRB engine 215 can monitor received participating site update information to ensure that the information is received by a particular time which may be specified in the notification or reminder sent to the participating site. If the update information for a site is received on time, the IRB module approves continuing site activity. If the update information is not received by the time specified, however, the IRB engine 215 can remove access privileges for that participating site. Privileges can be reinstated when the participating site complies with the update information requirements.

The reporting engine 220 can include one or more programmed reporting functions and statistical analysis algorithms. Exemplary reporting functions of the reporting engine 220 can include, but are not limited to, real-time management reports on screening rates and failures, study enrollment rates, and study follow-up rates. As information is uploaded to the system 200, the reporting engine 220 can generate real-time reports based on information gathered at that time. As such, sponsors and other authorized users of system 200 can identify problem sites earlier within the trial process and correct or replace those investigator sites in a timely manner.

The reporting engine 220 further can include other trial management related reporting functions such as subject recruitment, forms completion, and follow-up, each of which can be generated, viewed, and electronically forwarded to one or more network locations in real-time. Notably, access to reports can be strictly controlled through the use of appropriate passwords and/or other identifying information. Accordingly, authorized parties can generate, view, or otherwise access the reports and reporting functions from any suitable computer system having a network or Internet connection.

Another function of the reporting engine 220 can include automatic generation of textual summaries which include data elements entered on case reports that comprise a subject visit. This reporting function can save the physician time, provide an accurate accounting of the collected data, and allow the physician to print, electronically sign, and electronically file the summary if correct with the system 200. If errors are found, the physician can go back and correct any such errors before the summary is printed, signed, and filed. The end result is a comprehensive, easy-to-read, accurate medical summary that agrees with the clinical data in the study database. No further editing or review is needed. The clinical summary is complete when the patient visit is complete. Notably, as the present invention ensures that data is essentially clean and complete as entered, the system 200 has the ability to perform analysis in real time. While this may provide beneficial insight, safeguards can be built into the system 200 which preclude data analyses until statistically significant data samples are obtained.

AE management engine 225 can include or access AE forms which can be filled out online. The AE management engine 225 can link to needed clinical data or automatically request additional information depending on the type or classification of the AE reported. For example, particular forms and/or fields of forms can be associated with actions that can be automatically initiated upon completion of the electronic form. The AE electronic form, once submitted, can be stored in a data store, whether within the AE management engine 225 or within the centralized data store. The AE electronic form also can be sent electronically, for example using electronic mail, to one or more appropriate sponsor safety groups and principal investigator groups responsible for follow-up of AE's. The AE management engine 225 further can electronically submit appropriate documentation to the FDA.

As noted, the AE electronic form can be linked to one or more other data sources and/or initiate particular actions. For example, fields of the AE electronic form can be linked to electronic documents which provide further explanation. Similarly, fields can be linked to electronic mailing functions to send information to predetermined destinations. For instance, a response entered into a field, a check of a particular check box, and/or a selection of a particular drop down menu option can cause a notification or the form itself to be electronically sent to one or more destinations. It should be appreciated, however, that such functionality can be applied to any form used within system 200, and is not limited to AE electronic forms.

The EDC engine 230 can be configured to synchronize data and/or upload data from personal digital assistants (PDA's) or other portable data entry devices and/or communication devices such as laptop computers, portable phones, and the like. Accordingly, a physician and/or subject can enter data directly into such a PDA device and then upload the data to the system 200. Notably, the uploading and/or synchronization can be performed via a conventional wired communication link or via a wireless link using appropriate synchronization software. In some cases, for example depending upon the particular PDA used, the investigator site may be required to utilize software which facilitates such data synchronization or uploading.

Thus, the communications device can be communicatively linked to the system 200 via a cradle or other connection, including a local wireless connection, to a computer system in the investigator site which further facilitates data exchange through a network connection to the system 200. Notably, a wireless device also can be used which can transmit data over a longer range wireless communications link directly to a relay station or other receiving device that is not on-premises at the investigator site. The relay station and/or receiver can be communicatively linked to the system 200.

The EDC engine 230 further can coordinate the electronic transfer of information between other service providers. For example, when a subject enters a study, a message can be electronically forwarded to an external lab or other medical facility to create an appointment for the subject, or to advise that a lab sample is being sent. Identification information, critical to the linking of data generated from all sources, is automatically provided by the system 200 for the investigator site and follows the subject's data throughout the external data capture process, for example when test results or other clinical research data is received from external labs or testing facilities.

If the subject does not appear for a lab appointment, the EDC engine 230 can automatically notify the investigator site and/or the lab to rectify the situation. As soon as the lab data is collected, the data can be transmitted electronically to the EDC engine 230 which can store the data in the data store along with a report which is made available to the investigator site, a coordinating center, and the sponsor. The report can indicate the status of all subject visits pending, completed, and missed. This results in maximum patient follow-up and coordination of all study information, leading to an increase in study efficiency and accuracy.

The financial engine 235 can be configured to make payments to designated participant sites as may be required. The financial engine 235 can utilize a set of rules which allow for the automatic payment based upon any item of information that is tracked by the system 200. The rules further can specify time frames or other milestones during which the requirements for payment or reimbursement must be met. For example, the financial engine 235 can be configured to make automated payments to investigator sites at regular intervals or when one or more criteria of a payment schedule are reached. Such can be the case, for instance, when the investigator site accumulates or screens a particular number of study subjects. In another embodiment, the payment size can be based, at least in part, upon the number of subjects that the participant site has registered.

The financial engine 235 can be programmed to make payments based upon other rules such as when a given number of follow-up visits have been completed. The financial engine 235 can be configured to process and execute all reimbursements electronically and automatically as clinical data is captured. Electronic funds transfer can be made to the financial institution(s) of respective investigator sites on the day of required payment. Justification for payment can be sent identifying how many recruited subjects and approved follow-up visits have been reimbursed.

The financial engine 235 can be configured to generate compensation reports in real time for the investigator site or investigator and sponsor to view. Financial data further can be recorded by the financial engine 235 into the data store. Accordingly, either party may access the financial data within the data store or access a reporting function to identify how much has been earned at any time based upon the data that has been entered and/or generated within the system 200.

The audit engine 240 can log all, or selected transactions as specified by an administrator, for example, within the system 200. The audit engine 240 can stamp or annotate transactions between accessing users and the system 200 with supplemental information. For example, such transactions can be stamped with identification information indicating who, or which computer system, provided the information, timing information such as when the information was provided, source information indicating where the information originated, as well as how. Complete information on each transaction is preserved in the system 200, for example in the data store.

Responsibility for data, therefore, can be attributed to the person whose digital signature was used to access the system for that data entry function. The system 200 automatically creates an audit trail showing how the data was created, changed, and by whom. Audit trail techniques also can be used to monitor data management operations, i.e. by parties other than investigator sites. If so configured, the audit trail can automatically track all clinical trial operations.

The system 200 also provides for source document verification with respect to electronic documents using a variety of techniques. While this functionality is described with reference to the audit engine 240, source document verification also can be included or provided as part of the reporting engine 220 previously described. According to one aspect of the present invention, users can be presented with a repeat listing of data recently entered into the system 200. The investigator can review the electronic form and either accept the form or further edit the electronic form to correct any errors. According to another aspect of the present invention, a complete listing of all data entered for a subject visit can be presented for investigator review at the conclusion of a data entry session prior to further processing of the data. As activity within system 200 is tracked, the investigator having entered the data can be tasked with source document verification for all data provided by that investigator.

The system 200 can be configured to support controlled vocabulary standards such as MedDra and UMLS, data representation standards such as Extensible Markup Language (XML), Clinical Data Interchange Standards Consortium (CDISC) Operational Data Model (ODM) for representing clinical trials and accompanying data, and Health Level 7 (HL7) for representing clinical values. Other requirements such as from the Office for Human Research Protection (OHRP) as well as international and country specific requirements can be supported. The system 200 also can be configured to support the FDA Adverse Event Reporting System (AERS).

As noted, while information, such as the various forms and information described herein, can be stored in each respective processing engine or component, some or all of the data can be stored in a central data store. Each field of each case report form can be appropriately checked against all necessary elements on that same form, as well as against data from all other forms. Data from a completed case report form need not be accepted into the database until all fields contain valid, consistent, and complete information. The result is a smart case report form that eliminates invalid or missing data entering the data store.

For example, processing rules can be established on a per form and/or per field basis. In illustration, a field which is to receive a systolic blood pressure can be associated with a numerical range within which the entered number must fall to be considered a valid entry. Valid entries can be allowed to enter the data store. Invalid values or missing values can be eliminated or cause the system 200 to notify the user that the provided value was not valid and request another. Front-end validation substantially changes the data collection process and shifts the paradigm from data entry from written records to capturing correct electronic data initially. This paradigm shift in collecting data on a smart case report form reduces the number of erroneous values entering the database. Data queries from clinical trial monitors and, hence, monitoring overall, can be reduced.

Whether using a centralized data store or one or more distributed data stores, data can be encrypted for transfer over the Web or other communications network when sent to the system 200. The data is not retained at each investigator site locally. Rather, each investigator site can access their clinical data as needed as in any other study, but only over the Web during an online session.

Regarding protocol amendments, notifications can be posted to an access interface of the Web site and thus can be made available automatically at each investigator site the next time that site logs onto system 200. Accordingly, investigator sites automatically implement protocol changes enforced by the system 200.

The system 200 provides increased security with regard to the clinical trial process in that no study data or software need reside at an investigator site. All access to study information can be routed through, for example, a central server of system 200 by using appropriate access through a unique user identifier, password, and/or other user identifier. All transactions can be recorded within the system. This process maximizes protection of study information and limits access to only authorized users. Encryption of information for transfer between the investigator site, the central server, and/or any other entity accessing system 200 can be employed to provide increased security.

The change from a decentralized data capture process with minimal control and supervision of site study conduct to a centralized process where all site activities and all coordinating center activity can be audited in real-time fundamentally improves the relationship of site participants to the trial data.

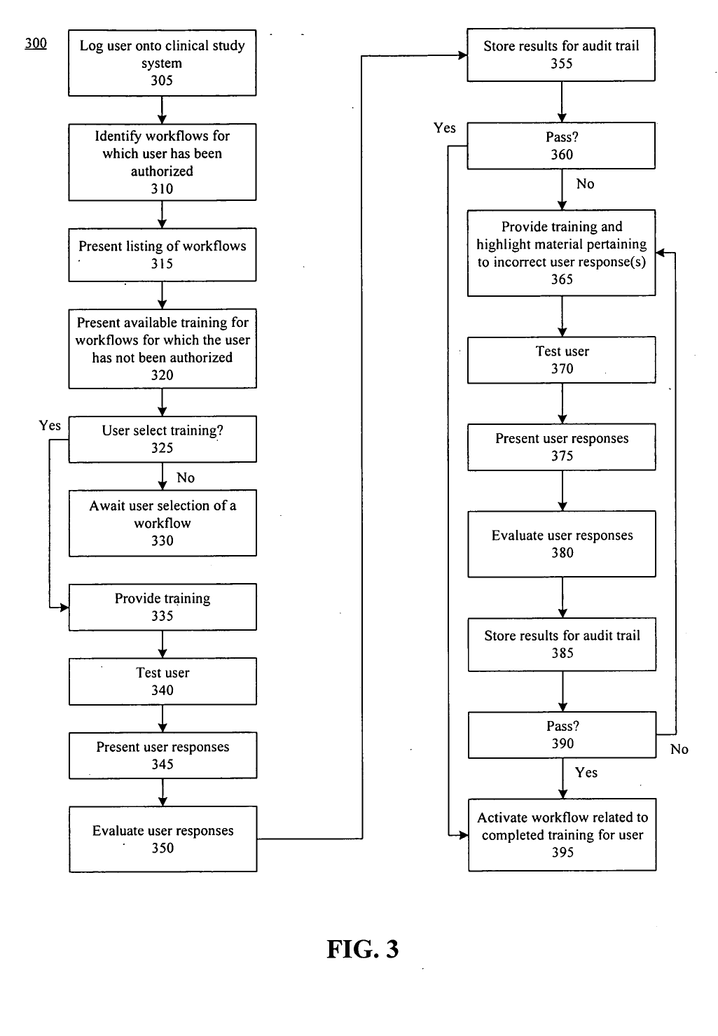

FIG. 3 is a flow chart illustrating a method 300 of training users for workflows and/or functions offered by the clinical study system for a clinical trial in accordance with another embodiment of the present invention. The method 300 can begin in step 305 where a user, for example a doctor or physician participating in a clinical trial, can log onto the clinical study system from a computer system at the physician's office, i.e. a participating site. The user can be authenticated using a username and password.

In step 310, the workflows for which the user has been authorized can be identified. That is, the clinical study system can maintain a user profile listing each feature or workflow of the clinical study for which the user has been authorized. A non-exhaustive listing of such features or workflows can include training, query management, clinical data capture, system configuration, and reporting functions. This user profile can be accessed once the user logs on, thereby allowing the clinical study system to identify any such workflows. In step 315, a listing of the workflows for which the user has been authorized to perform can be presented. This information can be presented, for example, upon a Web page that is sent to the user's computer system at the participating site.

In step 320, the workflows for which the user requires training can be presented. Such information can be sent to the user's machine in the form of a new Web page or as part of the previous Web page. Each of the presented workflows for which the user requires training cannot be accessed by the user until such time that the user successfully completes online training for the desired workflow.

In step 325, a determination can be made as to whether the user has selected training for a workflow. If not, the method can proceed to step 330 as the user desires to access a different functionality of the clinical study system. If the user selects training, a listing of different workflows or clinical trial activity for which training is provided by the system is presented to the user. The user then can select a particular workflow for which training is required and also for which the user wishes to begin training.

In step 335, the clinical study system can present training to the user. The clinical study system can provide training materials to the computer system of the user. The training materials can include text, pictures and/or graphics, video, and audio. For example, in one embodiment, the training materials can be provided as a series of one or more Web pages to be displayed sequentially and through which the user can navigate forward and backward.

In step 340, the user can be tested. That is, once the user has completed the training materials, a set of one or more questions pertaining to the training materials can be presented. In one embodiment, the questions can be presented as an online questionnaire with fields or selection mechanisms for receiving user responses. Responses to the questions can be captured by the clinical study system. In step 345, each question and response pair can be presented to the user. The user can choose to modify one or more responses or finalize the responses at this time.

In step 350, once finalized, the user responses can be evaluated. The user responses can be compared with a set of correct answers to the questions posed which are stored in the clinical study system. In step 355, the results of the user's online test can be stored in the central data store. A determination of whether the user passed the test or failed can be made in step 360 based upon the number of correct answers. This result also can be stored. If the user passed, the method can proceed to step 395. If not, the method can proceed to step 365. In any case, the user's profile can be annotated accordingly.

In step 365, the user can be presented with the training materials again. Notably, the training materials can include visual indications which highlight portions of the training material that are associated with the test questions the user answered incorrectly. This allows the user to focus more heavily upon those portions of the training materials. Thus, each question of a test can be correlated with a particular portion of the training materials thereby allowing such visual indicators to be provided when the training material is viewed or presented more than one time.

In step 370, the user can be tested again. Responses to each question can be received by the clinical study system. In step 375, the user responses and accompanying questions can be presented to the user for review. At this time the user can choose to go back and change one or more responses to the test questions prior to finalizing the responses. In step 380, the user responses can be evaluated after the user finalizes the responses. The results can be stored in step 385. A determination can be made as to whether the user passed or failed in step 390. This determination also can be stored in the user profile. If the user failed, the method can loop back to step 365 to repeat as necessary or until the user decides to quit and exit. If the user passed, however, the method can continue to step 395 where the workflow or clinical trial activity for which the training has been successfully completed can be activated for the user. The user's profile can be so annotated.

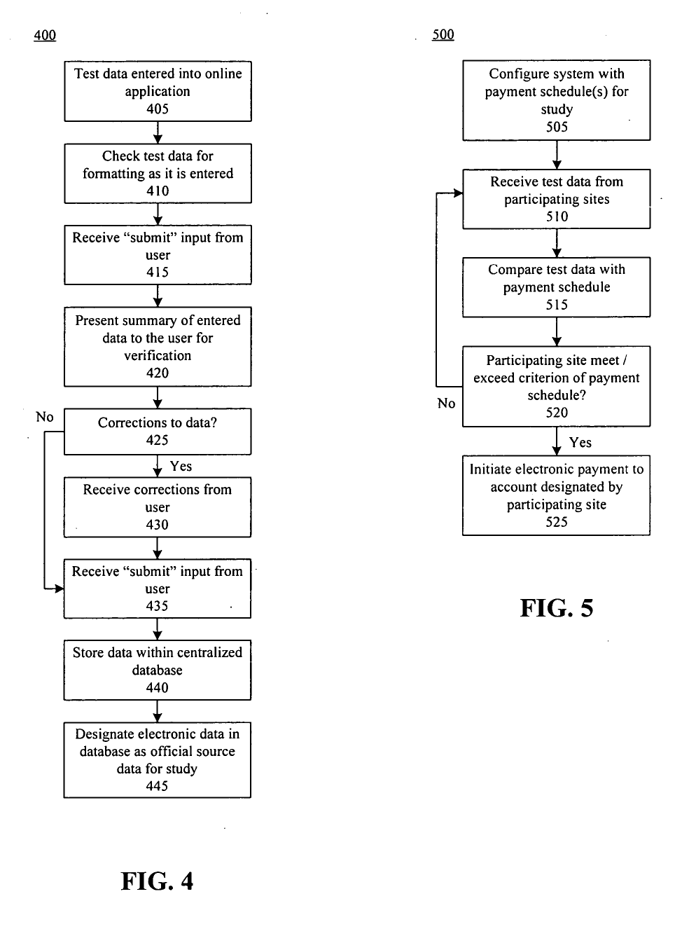

FIG. 4 is a flow chart illustrating a method 400 of electronic source document verification for use within a clinical trial in accordance with another embodiment of the present invention. The method 400 can begin in a state where a user has already logged onto the clinical study system. Accordingly, in step 405, the user, in this case a physician or other doctor, can enter clinical research data into a form provided by the clinical study system. As noted, one or more case report forms can be sent to the user's computer system thereby allowing the user to enter data pertaining to a patient's visit directly into the online form.

In step 410, the clinical research data can be checked for formatting errors. That is, dates and other fixed format data can be automatically verified for proper format and syntax. In step 415, a user input can be received indicating that the user has finished entering data for the particular patient and for this office visit. In step 420, the clinical study system can process the data and send a summary of the data to be displayed upon the user's screen. The data can be displayed as part of a Web page or the like. The display of the summary of the clinical research data entered by the user provides the user with an opportunity to verify the accuracy of the data just entered.

In step 425, the user can indicate whether any corrections are to be made to the data based upon a review of the summarized data. If so, the method can proceed to step 430. If not, the method can proceed to step 435. In step 430, one or more corrections to the data can be received. If not, the method can proceed to step 435. Notably, the data can be displayed in an editable format. For example, the summary can be editable, or another Web page can be displayed after the clinical study system receives an indication that the user wishes to modify the data. In step 435, the user can indicate that the information has been verified and is correct, or that the requisite modifications and/or corrections have been performed.

In step 440, the verified clinical research data can be stored in the centralized database of the clinical study system. In step 445, the clinical research data can be designated as official source data for purposes of the clinical study or trial in which the user was participating. This process ensures accuracy of the clinical research data and further eliminates the need for a paper documentation system.

FIG. 5 is a flow chart illustrating a method 500 of processing financial transactions within a clinical study in accordance with another embodiment of the present invention. The method 500 can begin in step 505 where the clinical study system is configured with one or more payment schedules. As noted, a payment schedule can be a generalized payment schedule or can be tailored for a particular participating site. In any case, the payment schedule can specify criteria for making payments to participating sites. The payment schedule can specify the amount of money to be paid, under what circumstances, and the account to which the money will be paid. For example, the payment schedule can specify the number of study participants to be signed up to receive a payment, which can be specified for increasing numbers of study participants, the number of follow-up visits to be performed with study participants to receive payment, which also can be specified for increasing numbers of follow-up visits, amounts of clinical study data that must be collected, minimal standards for the quality of the clinical study data, and the like.

In step 510, clinical research data can be received from a test site. Notably, while this task is illustrated as a single step, it should be appreciated that clinical research data is received throughout the course of a clinical trial and is constantly monitored and compared with study milestones. In step 515, the clinical research data received from the participating site can be compared with the payment schedule of the clinical study for which the data was submitted. In cases where a participating site is enrolled in more than one clinical trial or study, the data can be sent with an identifier that is associated with one of the clinical studies thereby enabling the clinical study system to associate and store the received clinical research data with the proper clinical study.

In step 520, a determination can be made, based upon the comparison of step 515, as to whether the participating site met or exceeded one or more criteria of the payment schedule. If so, the method can proceed to step 525. If not, the method can loop back to step 510 to repeat as necessary. In step 525, the clinical trial system can initiate a payment to an account designated by the participating site. The account can be specified within a profile for the participating site that is stored in the clinical study system, or if each participating site is associated with a payment schedule, such information can be specified within the participating site's payment schedule. In any case, the amount to be paid can be specified and/or whether any special accommodations are to be made to the participating site. This allows the clinical study system to make special arrangements for each participating site if so desired. In any case, the clinical study system can electronically contact a financial institution to initiate a transfer-of money from an account of the system to an account of the participating site.

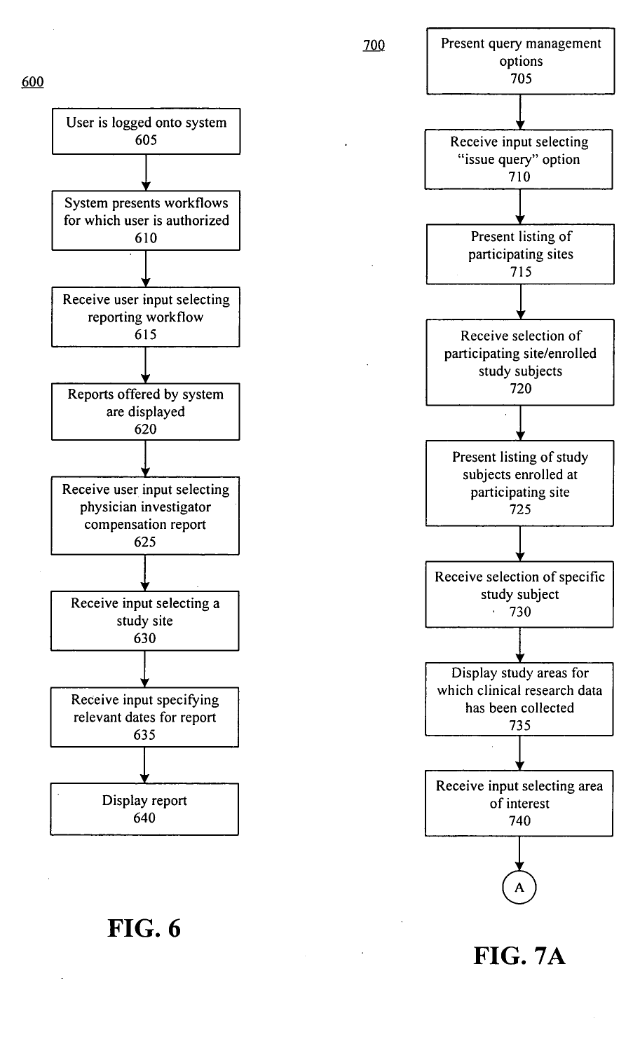

FIG. 6 is a flow chart illustrating an example of an investigator and/or sponsor selecting a particular workflow in accordance with one embodiment of the present invention. The method 600 can begin in step 605 where a user, for example a sponsor or an investigator, is logged onto the system. In step 610, the system presents the workflows for which the user has been authorized. In step 615, the system can receive a user input selecting a particular workflow for which the user has been authorized. For example, in this case the user has selected the reporting workflow.

In step 620, the various reports offered by the system can be presented to the user. A non-exhaustive listing of such reports can include, but is not limited to, a compensation report, an AE report, an enrollment report, and a subject summary report. It should be appreciated that each of the aforementioned reports can be provided for the system in general, or as a whole, or can be detailed or limited by site. In step 625, a user input selecting a physician investigator compensation report can be received by the system.

In step 630, if the user has the ability to enroll persons at more than one participating site, the user can specify the particular site for which the user wishes to obtain a report. Thus, this step is optional in the sense that if the user can only enroll study subjects at a single site, the system defaults to that site. In step 635, user input specifying the dates of interest or bounds for the report can be received. This information can be provided through an online form for example.

In step 640, the report can be displayed. The physician investigator compensation report can specify information such as the number of complete enrollments, the amount paid for the enrollments, the number of visits performed and the amount paid for those visits, as well as the number of screening failures by that site and the amount paid for those unsuccessful screenings. The report is generated using information stored in the system database and, therefore, is as current as the data.

It should be appreciated that if the user accessing the reporting workflow is a sponsor, the sponsor may access reporting functions across all investigators and/or participating sites. If the user is an investigator, however, the user will be limited to viewing financial reporting information relating to that investigator's compensation.

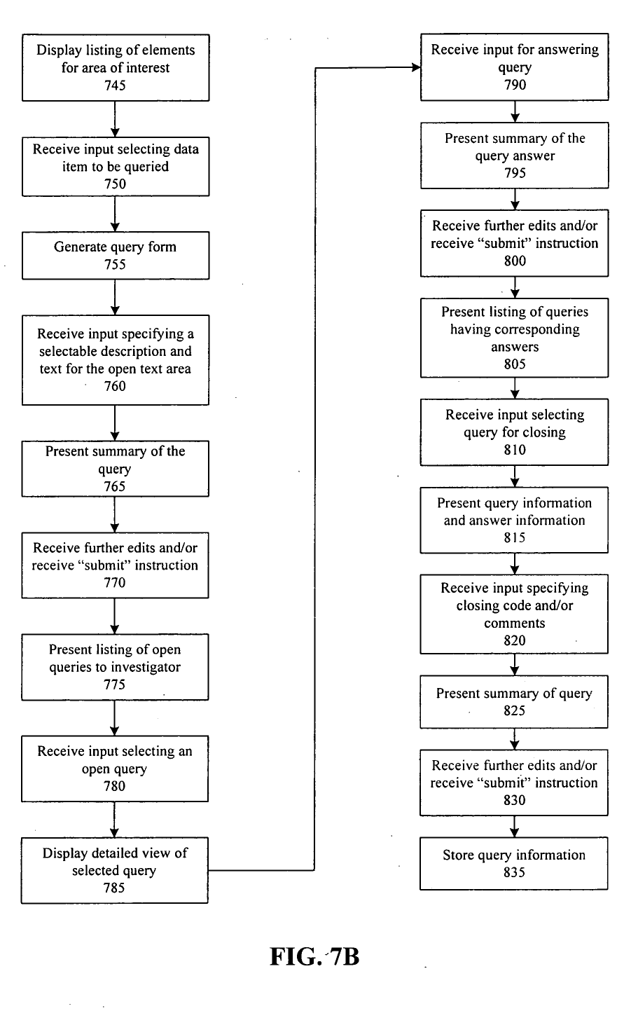

FIG. 7 is a flow chart illustrating an overview of the query functionality provided in accordance with another embodiment of the present invention. The query functionality can be provided as part of one of the engines previously discussed or can be incorporated into a query management engine. The method 700, while shown as a single flow chart, typically will be performed by more than one individual. As such, the method 700 serves as a general guide for illustrating the query functionality disclosed herein. Thus, the method 700 can begin in a state where a user has already been logged onto the system and has selected the query management workflow.

In step 705, query management options can be presented. Three options can be presented including issue a query, answer a query, and close a query. Typically, issuance of a query and closing of a query will be performed by a clinical study monitor. The answer option is usually performed by an investigator. In any case, queries are issued, answered, and then closed in that order.

In step 710, an input specifying the “issue query” option can be received by the system. A monitor can issue a query based upon a question relating to stored or captured clinical study data. In step 715, responsive to selecting the “issue query” option, a listing of participating sites can be presented. Next to each site name, can be an indication of the number of study subjects that are enrolled at that site. In step 720, a user input can be received from the monitor selecting a number of enrolled persons. The number of enrolled study subjects can be a hyperlink or other activatable icon or button. In step 725, upon selection of the number, the system can present a listing of the study subjects for the selected site. The monitor then can select any of the listed study subjects as the basis for the query in step 730. For example, the monitor can select an indicator next to the study subject for whom the monitor wishes to initiate a query.

Upon selecting a study subject, the various areas of the study for which clinical research data has been collected can be displayed for the selected study subject in step 735. In step 740, the monitor then can select the area of interest. The system in turn can display a listing of the data elements collected for the area of interest in step 745. The monitor then can select the data item to be queried in step 750. While the monitor can query more than one item of information in a single query, in another embodiment, each query can pertain to only a single item of information.

Once the item of information is selected by the monitor, in step 755, the system generates a query form. The query form includes at least two portions. The first portion is a series of one or more selectable options which describe the reason for initiating the query. Illustrative examples of possible selectable options can include, but are not limited to, “data is out of range”, “data does not match”, “data is missing”, “other”, or the like. The second portion is an open text area where the monitor enters text further describing the nature of the conflict to be resolved with the data item. In any case, the query can indicate the problem to be resolved. For example, if conflicting information within the centralized database exists, the monitor can issue a query to the appropriate investigator to resolve the conflicting data for the specified study subject. Notably, the audit information stored in the system allows the monitor to direct the query to a particular investigator such as the one having originally entered the data. In step 760, user inputs for the selectable portions of the query form and the open text area are received.

Once all of the information relating to the query has been provided, in step 765, the system presents a summary of the query to be issued. The summary is presented to the monitor for verification purposes before the query is actually issued. In step 770, the monitor views the query and, if accurate, indicates as much by selecting a “submit” button or other icon. Activation of the submit button indicates that the monitor accepts responsibility for the information being accurate. Still, the monitor can edit the query information prior to activating the “submit” option. Once verified, the query can be issued to the appropriate investigator.

Subsequently, the investigator for whom the query was issued can answer the query. Having been logged onto the system, the investigator can access the query management page and select the “open queries” option. Accordingly, in step 775, a listing of open queries or an indication that one or more open queries remain for the investigator to answer or resolve can be presented. Notably, queries can be presented to the investigator that initially entered the data item that is the subject of the query. Thus, using the audit trail and the user profiles, the issued queries can be directed and/or presented to the appropriate investigators. When viewing open queries, the investigator is presented with a listing of only those queries directed to him or her.

In step 780, the investigator can select an open query. In step 785, the query can be displayed in more detail and in a format that allows the investigator to enter an answer to the query. For example, the answer form can include a first portion having a series of one or more selectable responses, such as “data has been changed”, “data is correct as is”, “data has been added” or the like, and a second portion having an open text area for receiving textual input from the investigator. Thus, in step 790, input from the investigator is received that specifies one of the selectable options as well as text to be placed into the open text area.

Notably, in the case where information must be added or corrected in the centralized database, the system can utilize one or more safeguards. For example, if information is to be changed, an interface can be presented which displays the data element to be changed within the centralized database. The investigator is only permitted to continue answering the open query if the investigator first changes the data in the database. That is, the investigator cannot continue to complete an answer to the query until the data is corrected. Similar functionality can be used in cases where the investigator must add missing data.

Once answered, in step 795, the information entered by the investigator can be displayed in a summary format thereby allowing the investigator to review and verify the response just entered. In step 800, if the response is accurate, the investigator can select a “submit” option. Selection of the “submit” option indicates that the answer to the query is accurate and that the investigator accepts responsibility for the accuracy of the answer. Prior to selecting the submit option, however, the investigator can edit the information displayed.

Subsequently, the monitor can be logged back onto the system, or remain logged onto the system, as the case may be, and select an option to close queries. In step 805, the monitor can be presented with a listing of queries for which answers have been received from the investigator. In step 810, the monitor can select a query, view the answer provided by the investigator, and determine whether to close the query or keep the query open. Thus, in step 815, to close a query, the original “open query” information entered by the monitor followed by the investigator answer is presented. In step 820, the monitor enters a closing code indicating the acceptability and any action to be taken in response to the investigator's answer. For example, the code and/or monitor text response can indicate that no change need be made to the database or, alternatively, that the database has been properly updated.

In any case, as before, the monitor can be presented with a summary of the information entered in step 825. For example, the system can present the original query, the answer to the query, and the latest closing information entered by the monitor. In step 830, the monitor can edit the response, but by selecting the “submit” option, the monitor submits the information to the database, verifies the accuracy of the data, and accepts responsibility for the accuracy of the data. In step 835, the query information is stored in the system, for example as part of an audit trail, according to FDA guidelines as specified in 21 C.F.R. Part 11 guidelines.

The various steps and methods disclosed herein have been provided for purposes of illustration and are not intended as limitations of the present invention. The methods can be performed for one or more different participating sites and/or users as the case may be. Further, communications among the various entities described herein can be secured using any of a variety of different encryption mechanisms or other secured communications techniques.

The present invention provides for improved and more detailed monitoring of clinical research data. Data collection can be monitored in real time as information regarding the identity of the investigator that entered subject data and when can be preserved. As any transaction of the system can be preserved along with a corresponding audit trail, the present invention makes significantly more process information available than conventional paper based clinical study systems.

On-site monitoring of clinical data can be greatly reduced. Data and an investigator site's performance can receive more scrutiny using embodiments of the present invention. This increased level of monitoring requires less human effort and can be automated to a great extent. Accordingly, investigator sites can be audited for cause if necessary as such cause can be readily detected by the system, for example through an automated reporting function or detection of another metric or measurement which can be applied to the data as collected. Clinical trial monitors can view data as the data is accumulated and further electronically query investigator sites as to the accuracy of the data. The monitors can electronically ask the investigator site to confirm, correct, or acknowledge that data items may be incorrect.

It should be appreciated that while the inventive arrangements disclosed herein have been described with reference to managing and administering a single clinical trial, the present invention can be used to manage and administer more than one clinical trial simultaneously. That is, the clinical study system can be configured such that each clinical trial has its own set of interface pages and/or data processing components. For example, multiple instantiations of the clinical study system disclosed herein can be configured or the components of the system can be configured to directly manage and administer multiple clinical trials.

The present invention can be realized in hardware, software, or a combination of hardware and software. The present invention can also be realized in a centralized fashion in one computer system, or in a distributed fashion where different elements are spread across several interconnected computer systems. Any kind of computer system or other apparatus adapted for carrying out the methods described herein is suited. A typical combination of hardware and software can be a general purpose computer system with a computer program that, when being loaded and executed, controls the computer system such that it carries out the methods described herein.

The present invention also can be embedded in a computer program product, which comprises all the features enabling the implementation of the methods described herein, and which when loaded in a computer system is able to carry out these methods. Computer program or application in the present context means any expression, in any language, code or notation, of a set of instructions intended to cause a system having an information processing capability to perform a particular function either directly or after either or both of the following: a) conversion to another language, code or notation; b) reproduction in a different material form.

This invention can be embodied in other forms without departing from the spirit or essential attributes thereof. Accordingly, reference should be made to the following claims, rather than to the foregoing specification, as indicating the scope of the invention.

1. Within a centralized, online clinical study system, a method of coordinating a clinical study at a plurality of participating sites comprising:
logging a user onto the clinical study system;
comparing workflows of the clinical study that are available through the clinical study system with workflows for which the user has been authorized;
restricting access to workflows of the clinical study for which the user has not been trained; and
providing online training to the user for a workflow or clinical trial activity for which the user has not been trained.

2. The method of claim 1, said providing online training step comprising:
providing online training materials to the user, wherein the training materials are for the workflow for which the user has not been trained;
testing the user regarding the content of the online training materials;
determining whether the user passed the test; and
if so, authorizing the user to access the workflow for which the user has been trained.

3. The method of claim 2, wherein the user did not pass the test, said method further comprising:
presenting the online training material to the user, wherein selections of the training material that relate to at least one question the user answered incorrectly are visually indicated; and
retesting the user regarding the content of the online training materials.

4. Within a centralized, online clinical study system, a method of coordinating a clinical study at a plurality of participating sites comprising:
configuring the online clinical study system with a payment schedule of the clinical study;
receiving clinical research data from participating sites via a communications network;
comparing the clinical research data with the payment schedule of the clinical study; and
if at least one criterion of the payment schedule has been met or exceeded, initiating an electronic payment to an account designated by the participating site from which the clinical research data was received.

5. The method of claim 4, wherein the amount of the payment is established during said step of configuring the online clinical study system.

6. The method of claim 4, wherein the payment schedule specifies a time frame in which particular portions of clinical research data are to be received.

7. Within a centralized, online clinical study system, a method of coordinating a clinical study at a plurality of participating sites comprising:
receiving clinical research data pertaining to the clinical study;
receiving a user input indicating that the information is complete;
causing a summary of the clinical research data to be presented, whereby the user having provided the clinical research data can verify the accuracy of the clinical research data;
receiving an additional user input indicating that the clinical research data is accurate;
storing the clinical research data within a data store of the clinical study system; and
designating the stored clinical research data as official electronic source data for the clinical study.

8. The method of claim 7, prior to said step of receiving an additional user input, the method further comprising:
receiving a user input indicating that the clinical research data is inaccurate; and
receiving user corrections to the clinical research data.

9. A centralized, online clinical study system configured to coordinate aspects of a clinical study, said clinical study system comprising a training module configured to evaluate whether users have completed training for selected workflows for the clinical study that are available from the clinical study system, and provide training to registered users for the selected workflows.

10. The system of claim 9, further comprising a financial engine configured to determine when a participating site meets or exceeds a payment schedule of the clinical study based upon clinical research data received via a communications network from the participating site, and wherein said financial engine initiates a payment to an account designated by the participating site when the participating site meets or exceeds the payment schedule of the clinical study.

11. The system of claim 9, further comprising an audit engine configured to receive clinical research data and present a summary of the clinical research data to the user having provided the clinical research data for verification, wherein the audit system stores the clinical research data and designates the stored clinical research data as official source data for the clinical study.

12. The system of claim 9, further comprising an institutional review board engine configured to determine whether a potential test site has met requirements for participating within the clinical study.

13. The system of claim 9, further comprising a query management engine configured to open, close, and answer queries relating to items of clinical research data.

14. The system of claim 13, wherein the query management engine routes queries to investigators that originally entered the data item that is the subject of the query.

15. The system of claim 14, wherein the query management engine prevents a user from answering a query without first correcting the subject data item.

16. The system of claim 9, wherein the online clinical study system includes at least one case report form that can be transmitted over a communications link to a participating site computer system, and wherein at least one data entry field of the case report form is linked with a portion of a protocol of the clinical study explaining the at least one data entry field.

17. The system of claim 9, further comprising an adverse event management engine configured to electronically notify a third party of selected adverse events.

18. The system of claim 17, wherein the third party is the Food and Drug Administration.

19. A machine readable storage, having stored thereon a computer program having a plurality of code sections executable by a machine for causing the machine to perform the steps of:
logging a user onto a centralized, online clinical study system;
comparing workflows of a clinical study that are available through the clinical study system with workflows for which the user has been authorized;
restricting access to workflows of the clinical study for which the user has not been trained; and
providing online training to the user for a workflow or clinical trial activity for which the user has not been trained.

20. The machine readable storage of claim 19, said providing online training step comprising:
providing online training materials to the user, wherein the training materials are for the workflow for which the user has not been trained;
testing the user regarding the content of the online training materials;
determining whether the user passed the test; and
if so, authorizing the user to access the workflow for which the user has been trained.

21. The machine readable storage of claim 20, wherein the user did not pass the test, said method further comprising:
presenting the online training material to the user, wherein selections of the training material that relate to at least one question the user answered incorrectly are visually indicated; and
retesting the user regarding the content of the online training materials.

22. A machine readable storage, having stored thereon a computer program having a plurality of code sections executable by a machine for causing the machine to perform the steps of:
configuring a centralized, online clinical study system with a payment schedule of a clinical study;
receiving clinical research data from participating sites via a communications network;
comparing the clinical research data with the payment schedule of the clinical study; and
if the at least one criterion of the payment schedule has been met or exceeded, initiating an electronic payment to an account designated by the participating site from which the clinical research data was received.

23. The machine readable storage of claim 22, wherein the amount of the payment is established during said step of configuring the online clinical study system.

24. The machine readable storage of claim 22, wherein the payment schedule specifies a time frame in which particular portions of clinical research data are to be received.

25. A machine readable storage, having stored thereon a computer program having a plurality of code sections executable by a machine for causing the machine to perform the steps of:
receiving, within a centralized, online clinical study system, clinical research data pertaining to a clinical study;
receiving a user input indicating that the clinical research data is complete;
causing a summary of the clinical research data to be presented, whereby the user having provided the clinical research data can verify the accuracy of the clinical research data;
receiving an additional user input indicating that the clinical research data is accurate;
storing the clinical research data within a data store of the online clinical study system; and
designating the stored clinical research data as official electronic source data for the clinical study.

26. The machine readable storage of claim 25, prior to said step of receiving an additional user input, the machine readable storage further causing the machine to perform the steps of:
receiving a user input indicating that the clinical research data is inaccurate; and
receiving user corrections to the clinical research data.
